(19) United States
(12) Patent Application Publication
(10) Pub. No.: US 2013/0190310 A1
(43) Pub. Date: Jul. 25, 2013

(54) MIG6 AND THERAPEUTIC EFFICACY

(71) Applicants: David Sidransky, Baltimore, MD (US); Xiaofei Chang, Lutherville, MD (US)

(72) Inventors: David Sidransky, Baltimore, MD (US); Xiaofei Chang, Lutherville, MD (US)

(73) Assignee: THE JOHNS HOPKINS UNIVERSITY, Baltimore, MD (US)

(21) Appl. No.: 13/812,735

(22) PCT Filed: Jul. 26, 2011
(86) PCT No.: PCT/US11/45331

§ 371 Date: Apr. 11, 2013

Related U.S. Application Data
(60) Provisional application No. 61/367,696, filed on Jul. 26, 2010.

(51) Int. Cl. C12Q 1/68; C12Q 1/48

(52) U.S. Cl. CPC C12Q 1/6886; C12Q 1/485
USPC 514/234.5; 435/7.23; 435/6.12; 506/9; 514/266.4

(57) ABSTRACT

We identify markers capable of guiding the decision to incorporate epidermal growth factor receptor (EGFR) inhibitors, in particular EGFR tyrosine kinase inhibitors (TKIs), into chemotherapeutic regimens. Mitogen-inducible gene 6 (Mig6), a negative regulator of EGFR, is selectively upregulated during the development of resistance to the EGFR tyrosine kinase inhibitor (TKI) erlotinib, resulting in decreased EGFR phosphorylation. The ratio of Mig6/EGFR expression highly correlates with erlotinib sensitivity. A low Mig6/EGFR ratio correlates with a high response rate to gefitinib and a marked increase in progression-free survival for patients. The ratio of Mig6 to EGFR is a major predictor of biologic and clinical responses to EGFR inhibitors.

This invention was made using funding from the U.S. government. The U.S. government therefore retains certain rights under the terms of National Institutes of Health grants P50 DE019032, U01 CA084986 and R37DE012588.

### TECHNICAL FIELD OF THE INVENTION

This invention is related to the area of personalized medicine. In particular, it relates to predicting efficacy of anti-tumor drug therapy.

### BACKGROUND OF THE INVENTION

Selective small molecule tyrosine kinase inhibitors (TKIs) of EGFR, such as gefitinib and erlotinib were among the first targeted therapies developed for cancer. Some of these inhibitors have demonstrated benefit in select clinical settings, however, primary as well as acquired drug resistance eventually arises in most, if not all, treated patients (1-3). While primary somatic mutations in the tyrosine kinase domain of EGFR render tumors more sensitive to gefitinib and/or erlotinib (1, 4), and secondary mutations are associated with acquired drug resistance (3, 5), these genetic alterations are present in only a minority of patients who partially respond to treatment and are rare in tumors other than NSCLCs (2, 6-8). In order to be able to provide treatment selectively to those patients who do not harbor EGFR mutations but will nonetheless respond to TKIs, there is an urgent need to define the precise molecular mechanisms underlying resistance to EGFR-targeted TKIs, and to identify specific biomarkers capable of predicting therapeutic response.

Efforts have been made to correlate EGFR protein levels with the response to anti-EGFR therapy, however, the relationship between the two has been surprisingly poor (2, 8-10).

There is a continuing need in the art to predict which patients will respond and which patients will not respond to anti-tumor agents.

### SUMMARY OF THE INVENTION

One aspect of the invention is a method of predicting tumor resistance to an epidermal growth factor receptor (EGFR) inhibitor. A patient tumor sample is tested and expression level of mitogen inducible gene 6 (Mig6) and of EGFR are determined. The expression level of mitogen inducible gene 6 (Mig6) is compared to the expression level of EGFR. A ratio of Mig6 to EGFR lower than a predetermined cut-off value indicates sensitivity to the EGFR tyrosine kinase inhibitor and a ratio of Mig6 higher than the predetermined cut-off value indicates resistance to the EGFR tyrosine kinase inhibitor.

Another aspect of the invention is a method of predicting tumor resistance to an antibody to epidermal growth factor receptor (EGFR). A patient tumor sample is tested and expression level of mitogen inducible gene 6 (Mig6) and of EGFR is determined in the sample. The expression level of mitogen inducible gene 6 (Mig6) is compared to the expression level of EGFR. A ratio of Mig6 to EGFR lower than a predetermined cut-off value indicates sensitivity to the antibody and a ratio of Mig6 to EGFR higher than the predetermined cut-off value indicates resistance to the antibody.

Still another aspect of the invention is a method of stratifying patients on the basis of tumor characteristics. A patient tumor sample is tested and expression level of mitogen inducible gene 6 (Mig6) and of EGFR is determined. The expression level of mitogen inducible gene 6 (Mig6) is compared to the expression level of EGFR. The patient is assigned to a first group if a ratio of Mig6 to EGFR higher than the predetermined cut-off value is determined and the patient is assigned to a second group if the ratio is determined to be lower than the predetermined cut-off.

Yet another aspect of the invention is a method of predicting tumor resistance to an inhibitor of epidermal growth factor (EGFR), such as an anti-EGFR antibody or a tyrosine kinase inhibitor. A patient tumor sample isolated from a patient at a first time is tested and expression level of mitogen inducible gene 6 (Mig6) is determined. A patient tumor sample isolated from a patient at a second time is similarly tested and expression level of mitogen inducible gene 6 (Mig6) is determined. The second time is later than the first time. An increase in the expression level of Mig6 over time indicates an increase in the resistance of the tumor to the inhibitor.

These and other embodiments which will be apparent to those of skill in the art upon reading the specification provide the art with tools for assessing

### BRIEF DESCRIPTION OF THE DRAWINGS

FIG. 1A-1G. Mig6 is upregulated in an erlotinib resistant cell line which suppresses EGFR phosphorylation. FIG. 1A) Erlotinib-sensitive (SCC-S) and -resistant (SCC-R) cells were treated with erlotinib and cell viability was assayed. Values were set at 100% for untreated controls. FIG. 1B) Immunoblot analysis of protein expression in SCC-S and -SCC-R cell lines. FIG. 1C) SCC-S and SCC-R cells were treated with EGF at the indicated times and Mig6 protein expression was analyzed. FIG. 1D) Mig6 mRNA expression was examined by real-time quantitative PCR after EGF treatment at the indicated times. Mig6 mRNA expression was normalized to GAPDH expression. FIG. 1E) SCC-S and SCC-R cells were serum-stripped and stimulated with EGF for 60 min. Immunoprecipitation (IP) was performed against EGFR, followed by immunoblotting against Mig6 and EGFR. FIG. 1F) Densitometric quantification of Mig6 and EGFR. Data are presented as the ratio of Mig6/EGFR to indicate how many Mig6 molecules are associated with each EGFR molecule. All ratios are presented in relative arbitrary values. FIG. 1G) SCC-R cells were transfected with either scrambled siRNA or siRNA targeting Mig6 for 48 hrs. Cells were stripped in serum free medium overnight and stimulated with EGF for 15 or 60 min.

FIG. 2A-2G. Mig6 expression is upregulated by elevated phospho-AKT in SCC-R cells. FIG. 2A) Immunoblot analysis of phospho-AKT, total AKT, and loading control β-actin in SCC-S and SCC-R cells. FIG. 2B) SCC-R cells were treated with AKI (AKT1/2 kinase inhibitor, at 10 or 20 μM), U0126 (MEK1/2 inhibitor, at 10 or 20 μM), or DMSO (control) for 24 hrs and subjected to immunoblot analysis with indicated antibodies. 2C) SCC-R cells were treated with LY294002 (PI3K inhibitor, at 10 or 25 μM), rapamycin (mTOR inhibitor, at 1 or 2 μM) or DMSO (control) for 24 hrs and subjected to immunoblot analysis with the indicated antibodies. FIG. 2D) SCC-R cells were transfected with either scrambled siRNA or siRNA targeting PTEN for 48 hrs and subjected to immunoblot analysis. FIG. 2E) SCC-R cells were treated with 0.2 or 1 μM erlotinib (T0.2, T1, respectively) for 24 hrs, or pretreated with 0.2 or μM erlotinib for 30 min and then co-treated with 10 ng/ml EGF for an additional 24 hrs. Mig6 levels were then evaluated with immunoblot analysis. FIG. 2F) SCC-R cells were treated with 25 μM LY294002, 20 μM AKT1/2 kinase inhibitor, 2 μM rapamycin, or 20 μM U0126 for 24 hrs. Cells were then treated with 10 ng/ml EGF for 30 min to induce EGFR phosphorylation and subjected to immunoblot analysis. FIG. 2G) Densitometric analysis of phospho-EGFR/total EGFR. DMSO-treated samples were arbitrarily assigned a value of 1 and values of the remaining samples represent fold changes of phospho-EGFR per EGFR molecule. Note that fresh Mig6 antibody recognizes a nonspecific band above the Mig6 protein, which gradually disappears after antibody re-suing or recycling.

FIG. 3A-3K. Mig6 upregulation is associated with erlotinib resistance. 26 cancer cell lines were evaluated for total and tyrosine phosphorylated forms of EGFR and Mig6 by immunoblot analysis. β-actin or GAPDH were used as internal loading controls. Head and neck (FIG. 3A, with PC-3 as prostate), bladder (FIG. 3B), and lung (FIG. 3C) cancer cell lines were assayed. All cells were treated with indicated doses of erlotinib for 72 hrs and then viable cells were evaluated (FIG. 3D, FIG. 3E, FIG. 3F). Value was set at 100% for each vehicle-treated cell line. The exposure density of both EGFR and Mig6 blotted on the same membrane were quantified by densitometry and the value of Mig6/EGFR were plotted (FIG. 3G, FIG. 3H, FIG. 3I). Bladder (FIG. 3J) and lung cancer cell lines FIG. 3K) were stripped in serum-free medium overnight and treated with vehicle or 10 ng/ml EGF for 110 min, following pretreatment with vehicle or 0.1 μM erlotinib for 3 hrs. Cells were then subjected to immunoblot analysis for phospho-EGFR and total EGFR. β-actin was used as a loading control. Both shorter (p-EGFR-shorter) and longer (p-EGFR-longer) exposure times for phospho-EGFR are shown to provide more detail for each cell line.

FIG. 4A-4E. EMT is accompanied by increased Mig6, decreased EGFR phosphorylation and erlotinib resistance. A) Whole protein lysates were extracted from indicated cell lines and immunoblot analysis was performed with antibodies against E-cadherin and vimentin. B) H358 cells were treated with TGF-β1 or TGF-β3 for 1, 3, 7, 14 and 21 days. Immunoblot analysis was performed with antibodies against E-cadherin, vimentin, EGFR, p-EGFR, Mig6, and β-actin. C) Parental and TGF β-induced H358 cells were treated with erlotinib and cell viability was assayed. Values were set at 100% for untreated controls. D) H358 cells were treated with TGF-β1 TGF-β3 for 1, 3, 7, 14 and 21 days. Immunoblot analysis was performed with antibodies against AKT, p-AKT, p-Erk1/2, and β-actin. E) Cells induced with or without TGF-β1 for 21 day were treated with LY294002, U0126, or erlotinib for 24 hrs. Immunoblot analysis was performed with antibodies against Mig6 and β-actin.

FIG. 5A-5D. Mig6 expression correlated with erlotinib response in directly xenografted low passage lung and pancreatic tumors. FIG. 5A) Effect of erlotinib on growth of lung cancer xenografts (BML-1, -5, -7, and -11) was assayed and tumor growth curves displayed. BML-5 was sensitive to erlotinib. Data are plotted as mean±SEM. FIG. 5B) RNA from lung xenografts was extracted and real-time PCR of Mig6 was performed. Data are plotted as mean±SD after normalization with GAPDH. FIG. 5C) Whole protein lysates were extracted from lung xenografts and immunoblot analysis was performed with the indicated antibodies, FIG. 5D) Efficiency of erlotinib in inhibiting growth of lung and pancreatic tumor xenografts was displayed from most sensitive (left) to most resistant (right) as a bar graph. Tumor growth inhibition (TGI) indicates relative tumor growth of treated mice divided by relative tumor growth of control mice (T/C) in each case. Relative RNA expression of Mig6 in each tumor xenograft is displayed underneath the tumor growth inhibition bar as a heatmap. FC: fold change. Scale used was Log₂FC.

FIG. 6A-6D. Mig6/EGFR ratio correlates with the response of patients to gefitinib. FIG. 6A) Representative pictures of IHC staining against Mig6 and EGFR. FIG. 6B) Box plot of Mig6/EGFR ratio distribution across all 45 samples (from 0 to 4.33). FIG. 6C) The response of patients to gefitinib treatment. PD, progressive disease; SD, stable disease; PR, partial response. FIG. 6D) Kaplan-Meier survival curves showed that patients with low Mig6/EGFR ratio survived significantly longer than the high ratio patients and EGFR negative patients (Log-Rank test P=0.0112).

### DETAILED DESCRIPTION OF THE INVENTION

The inventors discovered that Mig6 is a major determinant of responsiveness to EGFR inhibitors. Additionally, tumor responsiveness to EGFR inhibitors can be predicted by the ratio of expression level of EGFR and Mig6. This ratio is a more powerful predictor than expression level of either gene alone. Thus these markers and their relative expression levels have clinical utility as predictive biomarkers.

Tumors which may be tested for EGFR inhibitor effectiveness include lung, head and neck, bladder cancer, pancreatic tumor, gastric tumors, colorectal cancer tumors, urothelial tumors, tumors of the liver, kidney, and bile duct, seminoma; embryonal cell carcinoma, choriocarcinoma, transitional cell carcinoma, adenocarcinoma, hepatoma: hepatocellular carcinoma, renal cell carcinoma; hypernephroma, cholangiocarcinoma, squamous cell carcinoma, epidermoid carcinoma and some malignant skin adnexal tumors. If a tumor may be resistant to EGFR inhibitor, economy as well as good clinical practice would suggest testing it prior to treatment for its EGFR: Mig6 ratio.

Measurement of expression levels of the two markers can be accomplished by any technique which yields quantitative assessment. These include without limitation, protein detection methods: immunohistochemistry, flow cytometry, Enzyme-Linked Immunosorbent Assay (ELISA), quantitative radio-immunoassay (RIA), and quantitative immunoelectrophoresis. Measurement of mRNA for the two markers can also be used, using any techniques which yield quantitative results. Such methods may include quantitative PCR, quantitative hybridization to a microarray, and digital PCR. Additional markers may be found which can be combined with the two markers to provide an improved assessment.

Samples which can be tested include any that contain tumor proteins or tumor nucleic acids. Typically the samples will be tumor tissue, whether surgically dissected tumors or biopsies. Xenografted tumor can also be used as a sample for testing. Tumor proteins or tumor nucleic acids may be shed into a body fluid and can be detected in the body fluid. Such body fluids may include stool, tears, saliva, sputum, bronchial lavage, urine, blood, lymph.

Although a cut-off value of 0.44 for the ratio of Mig6: EGFR has been found to discriminate well between sensitive and resistant tumors, it is possible that the cut-off could vary with different analytical techniques. The cut-off value could also vary in different tumors. New analytical techniques and new tumor types can be tested and validated in a population using samples and statistical techniques as described below or as known in the art. The reciprocal of the ratio can also be determined and values of 2.27 or lower of EGFR: Mig6 would provide the equivalent information.

The methods exemplified below provide a means of predicting resistance or sensitivity to an inhibitor treatment. The prediction may not be an absolute for an individual patient, but merely assigns the individual to a group which is resistant or sensitive. Any individual tumor and patient may have other characteristics or physiological or disease conditions which may mitigate the predictive power of the ratio. Prediction of sensitivity or resistance to a drug may also be called prognosis (determining survival, disease-free survival, or time before recurrence, for example) or theranosis. Additionally, the ratio may be used to stratify patients for example, for testing of additional drugs or therapeutic regimens. Stratifying assigns a patient to a group of patients that shares one or more characteristics. Here the group would have a similar ratio, either above or below a cut-off value. The group may be assigned a particular therapy based on the ratio. Or the groups may be subjected to a clinical trial and results analyzed on the basis of the groups.

Once a ratio is determined, an inhibitor can be prescribed to a patient, or an inhibitor can be administered to the patient. A prescription can be recorded in a medical chart, on a paper for transmission to a pharmacy, or electronically. A prescription can be transmitted to a pharmacy orally or telephonically. Administration of an inhibitor can be by a medical professional, by the patient, or by a third party. The mode of administration will be tailored for and appropriate to the particular inhibitor. Inhibitors may be administered by injection, by swallowing, by implantation, or other means as appropriate for the tumor and the inhibitor.

The assessments of ratio or absolute levels of expression of Mig6 may be performed at one or more time points for an individual patient. Time points for collecting samples may be spaced out by days, weeks, months, or years. A change in the ratio or absolute level of Mig6 may indicate a change in the sensitivity or resistance to an EGFR inhibitor. For example, if resistance develops in a tumor that is initially sensitive, the ratio may increase. The ratio may thus be used as an indication for discontinuing a treatment, or changing a treatment, or changing a dosage.

EGFR inhibitors include that those that are tyrosine kinase inhibitors (TKI) and those which are not specific enzyme inhibitors, such as antibodies which bind to EGFR. Suitable drugs include, without limitation, erlotinib (OSI-774, Tarceva), cetuximab (Erbitux), panitumumab (Vectibix), and gefitnib (Iressa). The inhibitors may be antibodies. The inhibitors may be multikinase inhibitors.

Our data suggest that the differential expression of Mig6, an ERBB family negative regulator, in human tumors is at least partially responsible for the weak association between wild-type EGFR protein expression levels and responsiveness to EGFR TKIs (2, 8-10). Although the erlotinib-sensitive tumors studied here generally displayed high EGFR levels, it was the activity of EGFR rather than its level of expression that most accurately predicted drug response. Supporting these findings, activation of the EGFR pathway has previously been reported to be the only reliable predictive factor of erlotinib responsiveness in pancreatic cancer patients (17, 18). In addition, when sensitive cancer cells are transformed to a lower phospho-EGFR phenotype, as is seen in an induced EMT-like transition, erlotinib resistance occurs (28). Our data also suggest that differential expression of the ERBB family negative regulator, Mig6, is a critical determinant of EGFR activity, and the extent to which cells utilize EGFR is a driving force for growth and survival. Cancer cells with EGFR overexpression could be erlotinib-resistant due to reduced dependence on EGFR signaling resulting from higher Mig6 expression levels. Neoplastic cells with a low Mig6/EGFR ratio may exhibit active EGFR signaling and sensitivity to EGFR TKIs, while those with a high Mig6/EGFR ratio frequently display reduced EGFR activity and resistance to EGFR TKIs.

Our findings also indicate that changes in baseline Mig6 expression may play an important role in acquired erlotinib resistance. Sensitive neoplastic cells may become resistant by acquisition of alternative growth factor pathways or by induction of Mig6 expression. In cell lines that acquired resistance to erlotinib we found that Mig6 upregulation was driven by markedly elevated basal PI3K-AKT activity. Since Mig6 functions to inhibit EGFR autophosphorylation, PI3K-AKT-mediated upregulation of Mig6 could negatively regulate signal input from EGFR once a cancer cell senses adequate growth and survival signals from alternative sources. This change would allow cells to shift their cellular phenotype towards a less EGFR-dependent state. Similar to our observation, a recent report on anti-ERBB2 trastuzumab therapy resistance demonstrated that all of the acquired resistant cell lines displayed reduced ErbB2 signaling with concomitant enhanced alternative RTKs signaling (29). However, it is worth noting that reduced basal EGFR activity is unlikely to be the sole determinant of acquired resistance to a variety of anti-EGFR agents in different laboratory models. Guix, et al., observed increased EGFR activity in A431 cells that acquired resistance to gefitinib (30). Increased EGFR phosphorylation was also seen in clones that developed resistance to anti-EGFR antibody cetuximab (31). At times cancer cells may generate resistance by increasing PI3K/AKT activity independent of EGFR, rather than by decreasing overall EGFR activity as reflected by the steady-state phosphorylation status (22, 30, 32). Although the mechanisms involved remain unclear, an association between EMT status and drug response has been consistently demonstrated in multiple cancer cells, including NSCLC (28, 33, 34), head and neck (35), pancreas, colorectal (36), and bladder (37) carcinomas. Interestingly, decreased EGFR activity has been previously observed in mesenchymal-like, erlotinib-resistant NSCLC cell lines (34). The mesenchymal-like cells from multiple tissue types studied here also displayed lower EGFR activity, along with higher Mig6 expression, suggesting that upregulation of Mig6 may contribute to the reduced EGFR activity observed in EMT. In addition, direct induction of EMT using TGF-β resulted in increased Mig6 expression, decreased EGFR phosphorylation, and the development of erlotinib resistance. A published TGF-β-induced EMT model using H358 cells similar to what we describe here confirmed that the induced cells exhibited kinase switching by aberrant expression of PDGFR and FGFR and loss of EGFR-dependence (28). Once the cells switched kinases for their survival and proliferation, they might become insensitive to EGFR inhibition.

One limitation of this study is that we were unable to knock down Mig6 in SCC-R to confirm the expected reversal of the cellular phenotype from resistant to sensitive to erlotinib when Mig6 expression is suppressed. For unknown reasons, depleting Mig6 in these cells, even with the resulting increased EGFR phosphorylation, induced cell cycle arrest (data not shown). However, others have previously demonstrated that mouse embryo fibroblasts (MEF) from Errfil−/− mice, driven by aberrantly active EGFR, proliferate more rapidly than those from the Errfil+/+ mice (38), while carcinogen-generated tumors that develop in Mig6 knockout mice are highly sensitive to gefinitib. Tumors in Errfil−/− mice regressed more than 50% in 1 week following initiation of gefitinib treatment, whereas those in control Errfil+/+ mice did not respond to gefitinib (15). In addition, a recent study demonstrated that depleting Mig6 per se in de novo cetuximab-resistant bladder cell lines rendered them responsive to the drug (39). These findings not only strongly support that Mig6 plays direct roles in resistance to multiple anti-EGFR drugs, but also provide additional biological basis for the observed sensitivity of human cancers which underexpress Mig6 to EGFR TKIs. When Mig6 is subsequently upregulated by EGFR-independent cellular events, such as the aberrant activation of PI3K-AKT, cancer cells are likely to develop resistance. Moreover, combining or augmenting treatments for further EGFR blockade are unlikely to have any further benefit as documented clinically (40). Our work highlights the importance of Mig6 expression in determining sensitivity to EGFR TKIs and identifies the potential clinical utility of the Mig6/EGFR ratio as a biomarker. The increased response rate and progression free survival observed here in patients with lung cancer whose tumors demonstrated a low Mig6/EGFR ratio are dramatic. The first IDEAL trial in NSCLC randomizing patients to gefinitib or placebo showed an overall difference of PFS of only 7 days (41), as compared to the median survival difference of nearly 100 days seen here. This finding further highlights the need to identify those patients most likely to respond to and benefit from therapy when treatment efficacy is evaluated. As an approach to personalized therapy, the expression levels of both EGFR and Mig6 could be examined in tumor cells, and the ratio of the 2 molecules could be used to select patients who are likely to benefit from anti-EGFR therapy. Subsequent increase in this ratio might indicate the development of drug resistance. Since Mig6 played a consistent role across multiple tumor types, the Mig6/EGFR ratio may be further clinically tested as a novel biomarker for predicting TKI response (and perhaps antibodies to EGFR as well) in diverse epithelial cancers. These findings provide a strong scientific foundation for validating the predictive accuracy of this biomarker in prospective clinical trials. Lastly, our work underscores the role of negative regulators of receptor RTKs in cellular utilization of these receptors and should be taken into consideration for drug response evaluation of any molecular targeted therapies to other RTKs.

The above disclosure generally describes the present invention. All references disclosed herein are expressly incorporated by reference. A more complete understanding can be obtained by reference to the following specific examples which are provided herein for purposes of illustration only; and are not intended to limit the scope of the invention.

### Example 1

### Materials and Methods

### Compounds and Reagents

Erlotinib (OSI-774, Tarceva) was purchased from Johns Hopkins University Hospital Pharmacy. LY294002 and U0126 were obtained from Cell Signaling Technology, Inc. (Beverly, Mass.). EGF was purchased from BD Pharmingen (San Diego, Calif.). All other chemicals were purchased from Sigma (St. Louis, Mo.), except where otherwise indicated. All chemicals and growth factors were dissolved in recommended vehicle as instructed by the manufacturers.

### Cell Lines

The human NSCLC cell lines (H226, H292, H358, H1838, A549, Calu6, H460, H1703, H1915, H1299, Calu3, H1437, and H23), human bladder cancer cell lines (5637, SCaBER, UMUC-3, T24, HT-1376 and J82), and human head and neck squamous cell carcinoma (HNSCC) cell line FaDu were obtained from American Type Culture Collection (ATCC). BFTC-905 was obtained from German Collection of Microorganisms and Cell Cultures (Braunschweig, Germany). Cells were maintained in a humidified atmosphere containing 5% CO2 at 37° C.

### Establishment of Acquired Resistance to Erlotinib

Drug resistant cell lines were generated via a process of slowly escalating exposure to erlotinib, as reported previously (16). SCC-S is used to designate the parental UM-SCCI cells exposed to DMSO, and SCC-R refers to the erlotinib resistant clone.

siRNA Transfection

Mig6 siRNA was synthesized and purchased from invitrogen (Carlsbad, Calif.) according to published sequences (15). PTEN siRNA was obtained from Cell Signaling Technology, Inc. (Beverly, Mass.), and EGFR siRNA was purchased from Santa Cruz Biotech (Santa Cruz, Calif.). Cells were plated in either 6-well or 96-well plates and transfected with the indicated siRNA using RNAiMAX transfection reagent (Invitrogen, Carlsbad, Calif.) according to the manufacturer's instructions. Cells were subjected to western blot analysis or viability assay 72 hrs post-transfection, unless otherwise stated.

### Antibodies and Immunoblot Analysis

Antibodies against EGFR, phospho-tyrosine (P-Tyr-100), phospho-EGFR (Tyr1068), phospho-HER2/ErbB2 (Tyr1248), AKT, phospho-AKT (Ser473), p44/42 MAPK, (Erk1/2), phospho-p44/42 MAPK (Erk1/2) (Thr202/Tyr204), and PTEN were obtained from Cell Signaling Technology, Inc. (Beverly, Mass.). Monoclonal anti-P-Actin antibody was obtained from Sigma (St. Louis, Mo.). Polyclonal anti-Mig6 antibody was a generous gift from Dr. Ferby (15). When appropriate, cells were cultured in serum free medium overnight, pretreated with the indicated inhibitors for 3 hrs or 2.4 hrs, and the treated with 10 ng/ml EGF for 10 or 30 min. Equal amounts of protein were mixed with Laemmli sample buffer, run on 4-12% NuPAGE gels and transferred to nitrocellulose membrane (Bio-Rad Laboratories, Hercules, Calif.). The membrane was probed with primary antibody followed by HRP-conjugated appropriate secondary antibodies (Santa Cruz Biotech, Santa Cruz, Calif.), and detected by enhanced chemiluminescence (ECL, GE Health Care, Piscataway, N.J.).

### Immunoprecipitation Analysis

SCC-S and SCC-R cells seeded in 100-mm Petri Dishes (Corning Inc., Corning, N.Y.) were serum-stripped overnight followed by treatment with vehicle or 10 ng/ml EGF for 60 min. Cells were washed with PBS and lysed using TRITON-X lysis buffer (50 mM Tris-HCl, pH 7.4; 150 mM NaCl, 1 mM EDTA; 1% TRITON-X100) containing protease inhibitors (Roche Diagnostic. Systems, Branchburg, N.J.) and phosphatase inhibitor cocktail (Sigma-Aldrich, St Louis, Mo.). Lysates were pre-cleaned with Protein A-Agarose beads (Santa Cruz Biotech, Santa Cruz, Calif.) and then incubated overnight at 4° C. with EGFR IP-specific antibody. Immune complexes were precipitated with protein Protein A-Agarose beads for an additional 4 h at 4° C., and then the nonspecific bound proteins were removed by washing the beads with lysis buffer five times at 4° C. The beads were loaded in Laemmli sample buffer directly onto the gel and analyzed by immunoblotting with anti-Mig6 and anti-EGFR antibody.

### Reverse Transcription (RT) and Real-Time PCR

RNA was extracted using Trizol (Invitrogen, Carlsbad, Calif.) followed by RNAeasy kit cleanup (Qiagen, Valencia, Calif.). RNA was reverse transcribed to cDNA using Superscript III (Invitrogen) which was then used as a template for real-time PCR. Gene products were amplified using iTaq SYBR green Supermix with Rox dye (Bio-Rad Laboratories, Hercules, Calif.). All reactions were performed in triplicate, with water controls, and relative quantity was calculated after normalizing to GAPDH expression. Expression of Mig6 mRNA relative to GAPDH was calculated based on the threshold cycle (Ct) as 2−Δ(ΔCt), where Δ(ΔCt)=ΔCtMig6−ΔCtGAPDH.

### Cell Viability and Drug Sensitivity Assay

Cells were plated at a density of 3000/well in 96-well plates. The following day, cells were treated with 0, 0.01, 0.033, 0.1, 0.33, 1, or 3.3 μM erlotinib for an additional 72 hrs. Cell viability was subsequently assayed using Calcein AM (Invitrogen), Fluorescence signals generated as a result of Calcein AM cleavage by viable cells were read by a Molecular Devices plate reader (Sunnyvale, Calif.) using an excitation frequency of 480 nm, and an emission frequency of 535 nm.

### Microarray Analysis

RNA was extracted from SCC-S and SCC-R and Affymetrix arrays were used for gene expression profiling. We used GeneChip Human Genome U133A 2.0 Arrays containing >22,000 probe sets for analysis of >18,400 transcripts, which include ˜14,500 well-characterized human genes. Probe preparation and hybridization were performed following manufacturer's instructions. Digitized image data were processed and normalized using the GeneChip software (version 3.1) available from Affymetrix.

### Xenograft Generation in Mice and Erlotinib Treatment.

The xenografts were generated and erlotinib treatment was performed as published previously (17, 18). Relative tumor growth inhibition (TGI) was calculated as the relative tumor growth of treated mice divided by relative tumor growth of control mice (T/C). The animals were maintained in accordance to guidelines of the American Association of Laboratory Animal Care and the research protocol was approved by the Johns Hopkins University Animal Use and Care Committee.

### Immunohistochemistry (IHC) Staining for Mig6 and EGFR

IHC were performed using an automated stainer (Dako Inc., Carpinteria, Calif.). Anti-Mig6 antibody was purchased from Sigma, and anti-EGFR were ordered from Dako (Carpinteria, Calif.). Tissue processing, deparaffinization, antigen retrieval and IHC staining were performed as directed by the manufacturer. Briefly, staining was performed by serially incubating tissue sections in Methanol/3% H2O2 (15 min), PBS, serum free protein (block) (7 min), rabbit anti-Mig6 or EGFR antibody (90 min at 22° C.), PBS (rinse), biotinylated secondary antibody (DAKO) (30 min at 22° C.), PBS, streptavidin-HRP (DAKO) (30 min at 22° C.), and PBS. Staining was visualized with 3,3′-diaminobenzidine (DAB) tetrahydrochloride (Zymed, Carlsbad, Calif.).

### Patient Selection

Formalin-fixed, paraffin-embedded (FFPE) tumor tissue samples were obtained from patients with advanced non-small cell lung carcinoma treated with gefitinib or erlotinib at The University of Texas M. D, Anderson Cancer Center between May 1999 and December 2004 (19). There were 45 samples available which were all included in this study. All tumor specimens were histologically classified according to the WHO classification for lung cancer by an experienced thoracic pathologist (I. I. W.) (20). Clinical response was graded according to the Response Evaluation Criteria in Solid Tumors (19, 21),

### Statistical Analysis

Student t-tests were used tier statistical analysis between two groups. All P values are based on two-sided. The significance level was defined as 0.05. Survival analysis was performed using Kaplan-Meier model and significance was determined using a two-sided log-rank test as well as Wilcoxon test. All statistical analyses were performed using SPSS.

### Example 2

### Acquired Resistance to Erlotinib is Associated with Upregulation of Mig6 Expression and Decreased EGFR Activity

A possibility that is commonly overlooked is that EGFR expression may be uncoupled from its activity via negative feedback regulators of EGFR family receptor tyrosine kinases (RTKs). Among these negative regulators, the multiadaptor protein mitogen-inducible gene 6 (Mig6, also known as RALT. ERRFI1 or Gene 33), plays an important role in signal attenuation of the EGFR network by blocking the formation of the activating dimer interface through interaction with the kinase domains of EGFR and ERBB2(11-14). Mig6 knockout (Errfil−/−) mice exhibit hyperactivation of endogenous EGFR, resulting in hyperproliferation and impaired differentiation of epidermal keratinocytes. In addition, carcinogen-induced tumors in Errfil−/− mice are unusually sensitive to the EGFR TKI gefitinib (15).

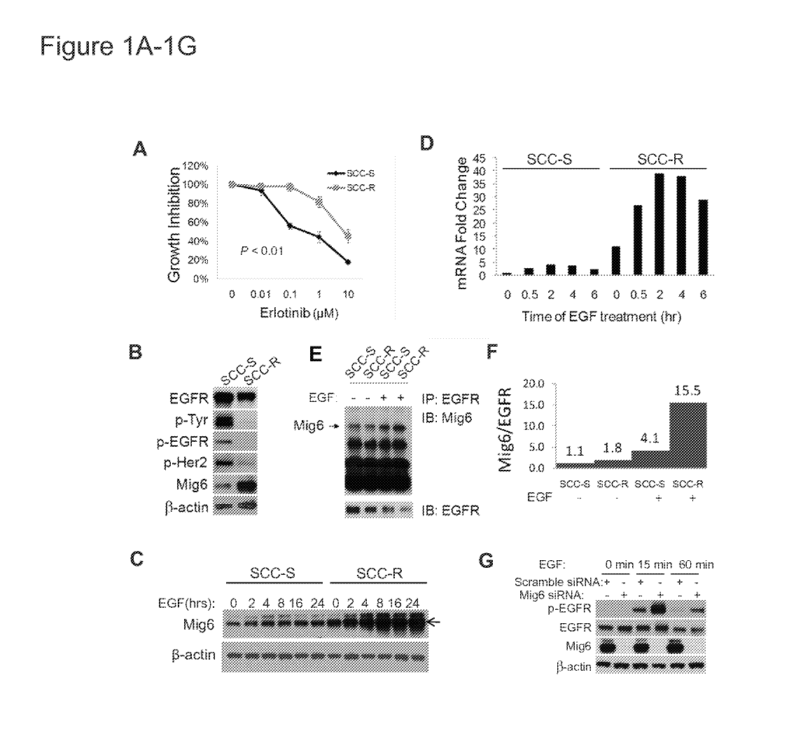

Erlotinib-resistant (SCC-R) and erlotinib-sensitive (SCC-S) isogenic cell lines were generated via chronic exposure of human head and neck squamous cell carcinoma UM-SCC SCC1 cells to either erlotinib or DMS( )(vehicle control). The IC50 of SCC-R cells was >10 times higher than that seen with SCC-S cells (FIG. 1A). Comparing the expression and basal activity of EGFR in SCC-S and SCC-R cell lines we found that the level of phosphorylated EGFR was markedly and disproportionally decreased in SCC-R cells (FIG. 1B). This apparent uncoupling of EGFR protein expression and activity in resistant cells was associated with a relatively higher expression of the endogenous ERBB family negative regulator, Mig6 (FIG. 1B). While treatment with EGF induced a rapid, sustained increase in Mig6 in both cell lines, Mig6 expression remained markedly higher in SCC-R cells as compared to SCC-S cells (FIGS. 1C and 1D). In addition, more Mig6 was found associated with EGFR in SCC-R cells (FIG. 1E). Densitometric quantification showed an almost four-fold increase in the level of Mig6 associated with EGFR in SCC-R cells after ligand stimulation as compared to SCC-S cells (FIG. 1F), indicating that the overexpressed Mig6 present in SCC-R cells was functionally active. Mig6 knockdown in SCC-R cells resulted in an increase of EGFR phosphorylation in response to treatment with EGF (FIG. 1G). These results suggest that the hypophosphorylation of EGFR Observed in SCC-R cells is due to excess binding of Mig6.

### Example 3

### Mig6 Upregulation in Erlotinib-Resistant Cells Line is Due to Activation of AKT

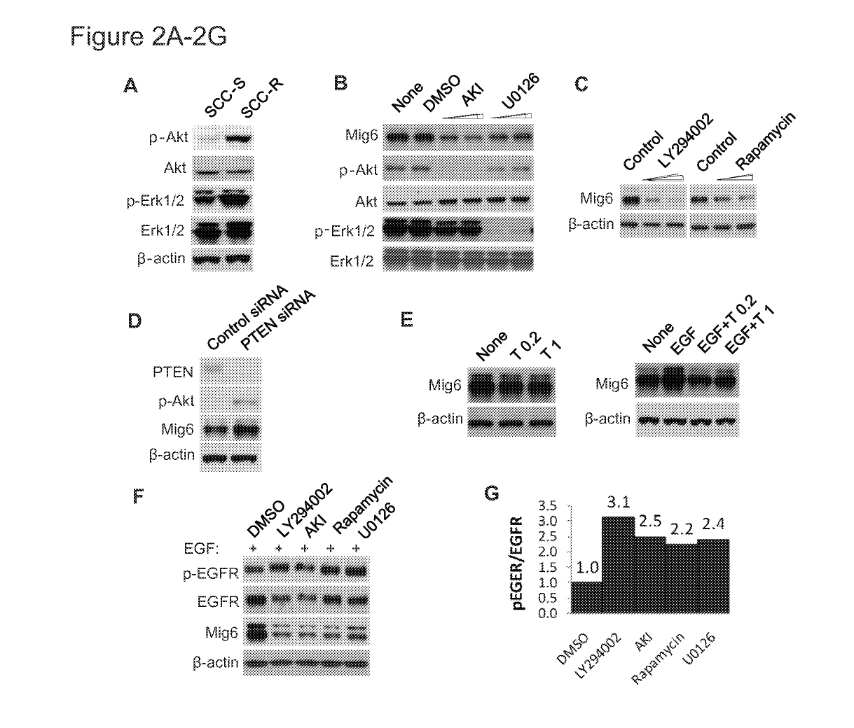

EGFR-independent activation of the phosphatidylinositol 3-kinase (PI3K) pathway has frequently been seen in t cells that develop resistance and is thought to confer resistance to EGFR TKIs (22, 23). We also observed that the basal phosphorylation level of AKT was higher in SCC-R cells than their sensitive counterparts (FIG. 2A). Microarray analysis revealed that multiple AKT ligands, including IGFR, PDGFR and FGFR, as well as upstream growth factor receptors, were significantly upregulated in SCC-R as compared to SCC-S cells (data not shown). It has previously been shown that Mig6 is regulated by the MEK/Erk pathway (24) and we did find higher Erk1/2 phosphotylation in SCC-R cells (FIG. 2A). We sought here to determine whether the PI3K pathway was also involved in regulating the basal expression level of Mig6 in SCC-R cells. Treatment of SCC-R cells with either an AKT1/2 kinase inhibitor (AKI) or a MEK inhibitor (U0126) decreased expression of Mig6 in association with the specific inhibition of each targeted pathway (FIG. 2B). Likewise, treatment of SCC-R cells with the PI3K inhibitor, LY294002, and the mTOR inhibitor, rapamycin, also decreased Mig6 expression (FIG. 2C). Conversely, direct activation of the PI3K-AKTpathway via RNAi-mediated silencing of PTEN expression resulted in an increase in Mig6 expression (FIG. 2D). In keeping with the role of EGFR-independent growth factor receptors in activating PI3K-AKT-mediated upregulation of Mig6, treatment of SCC-R cells with erlotinib produced only a slight decrease in basal Mig6 expression (FIG. 2E), even though erlotinib could completely abolish EGF-induced Mig6 upregulation (FIG. 2E). Furthermore, exposure to each inhibitor (LY294002, AKI, rapamycin, or U0126) increased the ratio of phospho-EGFR to EGFR (FIGS. 2F and 2G), consistent with the role of Mig6 in determining EGFR activity. It is worthy to note here that EGF ligand treatment was used to boost the signal detection since the basal EGFR phosphorylation level is below detectable level of this particular antibody. These data indicate that upregulation of PI3K-AKT-mTOR by alternative growth factor receptors promotes Mig6-mediated inhibition of EGFR activity, enabling EGFR-independent growth of tumor cells and rendering them insensitive to EGFR-targeted TKIs. Note that fresh Mig6 antibody recognizes a nonspecific band above the Mig6 protein, which gradually disappears after antibody re-suing or recycling.

### Example 4

### Mig6 Upregulation is Associated with Erlotinib Resistance in Cancer Cell Lines of Different Tissue Origins

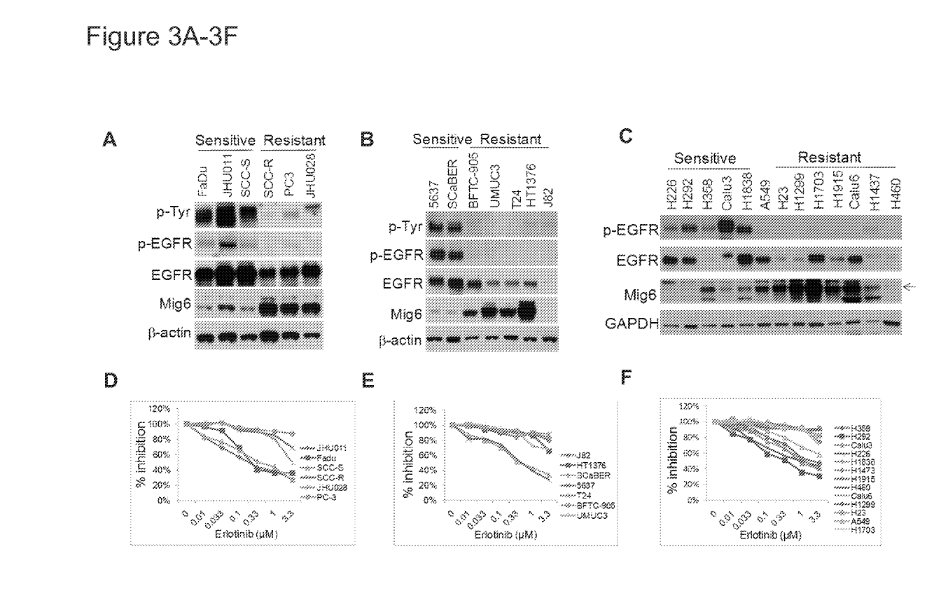

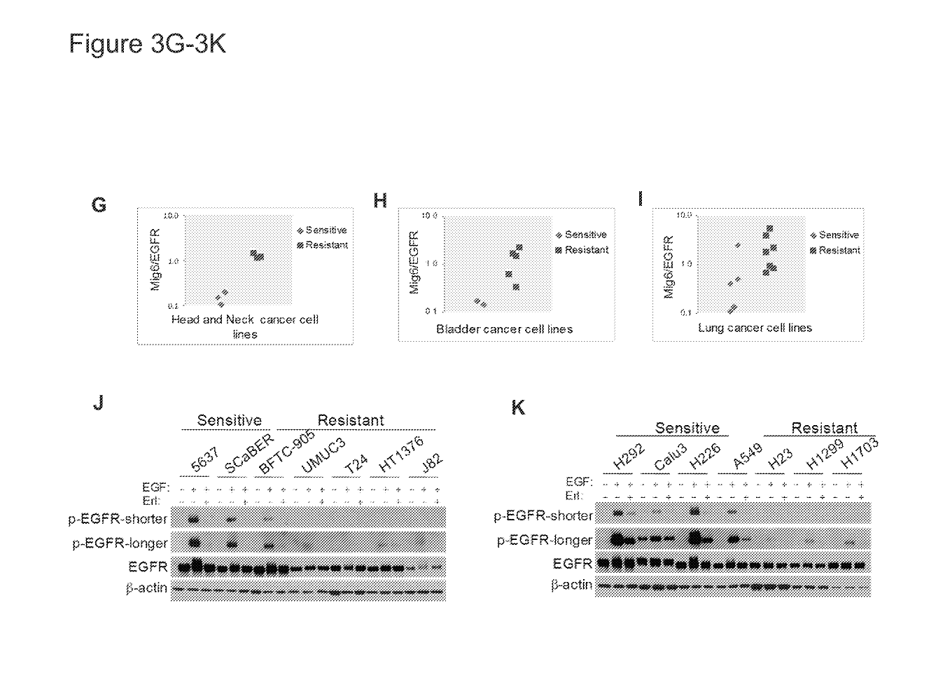

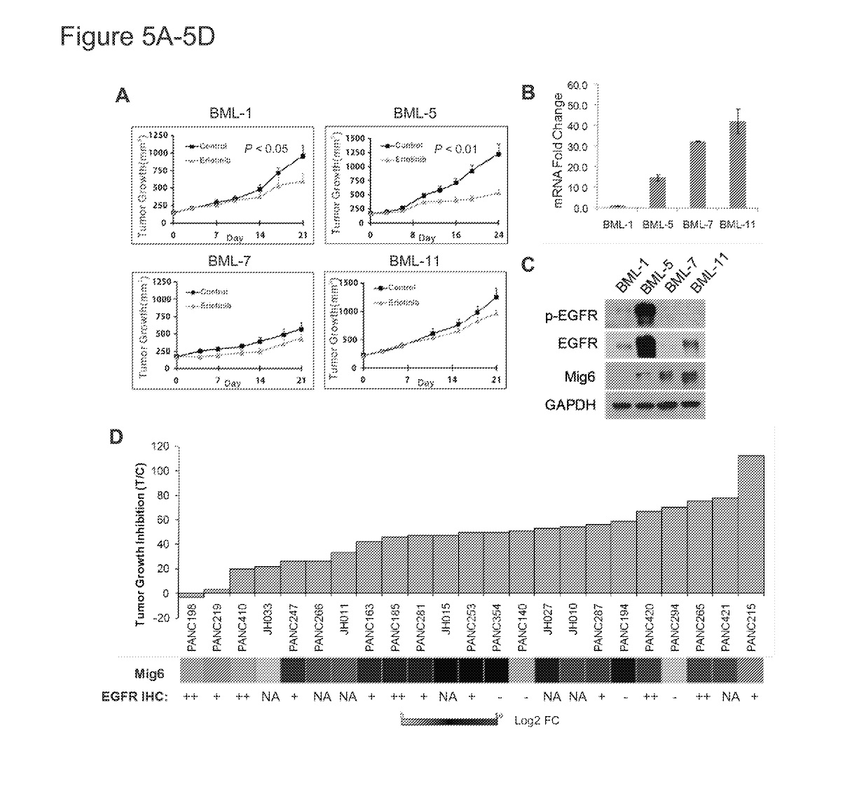

We next investigated Mig6 expression and EGFR activity in panels of cancer cell lines. At the maximum tolerated and currently used dose of erlotinib (150 mg per day), steady-state serum concentrations range between 0.33 to 2.64 μg/mL with a median of 1.2.6±0.62 μg/mL, or 2.9 μM (25). Because 90% of erlotinib is bound to serum proteins, the free drug concentration is approximately 0.3 to 1 μM. Therefore, for this study cells were defined as erlotinib-sensitive when significant cell growth inhibition (IC₅₀) vas observed at a concentration of erlotinib less than or equal to 1 μM, while cells that failed to undergo such growth inhibition were considered erlotinib-resistant. Lung cancer cell line A549 was considered intermediate-resistant based on its erlotinib response curve. Our data indicated that higher Mig6 expression was strongly correlated with lower levels of EGFR phosphorylation and erlotinib resistance in 6 of 6 head and neck and prostate cancer cell lines assayed (FIGS. 3A and D). Similar results were also observed in 17 of 20 bladder (FIGS. 5B and E) and lung cancer cell lines (FIGS. 3C and F). The exceptions to this pattern (J82-bladder cancer cell line, H1437 and H460-lung cancer cell lines) all showed low levels of Mig6, yet displayed an erlotinib-resistant phenotype. In each of these cases, the cells displayed very low basal EGFR expression when compared to their erlotinib-sensitive counterparts. Thus, across the cell lines tested, the ratio of Mig6 to EGFR, appeared to be a more reliable predictor of tumor cell response to erlotinib than the absolute expression of either protein alone (FIGS. 3G, H and I).

The association between high Mig6/EGFR ratio and erlotinib resistance suggests that tumor cells that have low EGFR, activity will be largely unresponsive to EGFR TKIs. In this situation, the resistance of tumor cells to EGFR inhibition results from the functional irrelevance of EGFR as opposed to the inability of these agents to inhibit basal or ligand-induced EGFR activity. To test this hypothesis, bladder and lung cancer cell lines were exposed to vehicle or erlotinib prior to treatment with EGF. EGF induced heavy EGFR phosphorylation in all sensitive cell lines, while only light phosphorylation was observed in the erlotinib-resistant cell lines tested (FIGS. 3J and K). This finding suggests that elevated levels of Mig6 in erlotinib-resistant lines may impair basal as well as ligand-induced activation of EGFR. Importantly, erlotinib was able to effectively block ligand-induced EGFR phosphorylation in all cell lines tested, indicating that the ability of erlotinib to block EGFR activation was not impaired even after cells developed resistance to its growth inhibitory effects.

### Example 5

### Epithelial Mesenchymal Transition (EMT) is Accompanied by Increased Mig6 Expression, Decreased EGFR Phosphorylation and Erlotinib Resistance

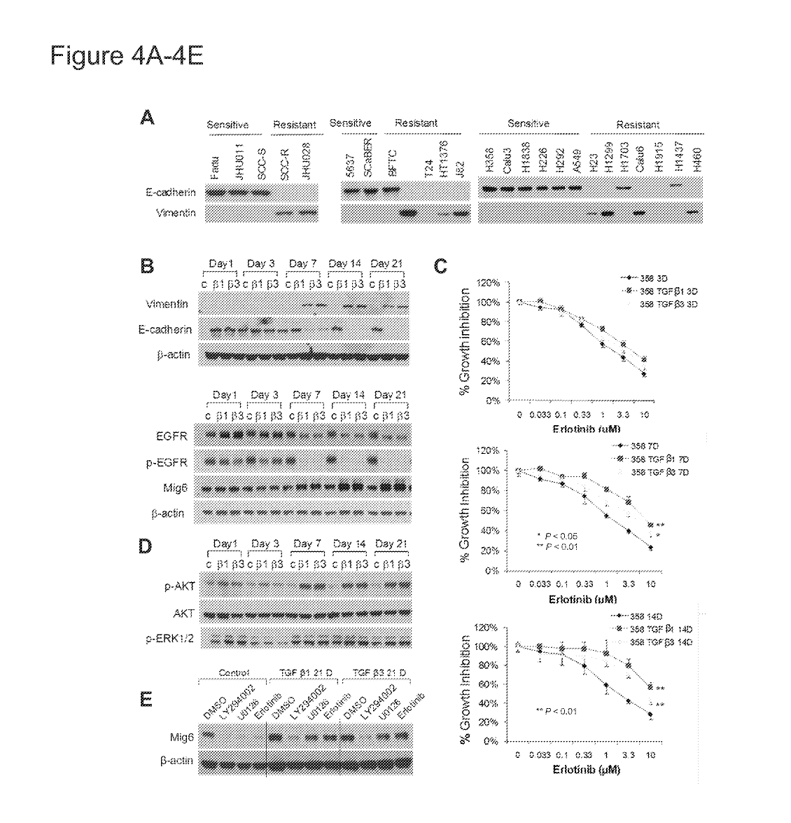

EMT has previously been demonstrated to predict resistance to erlotinib or gefitinib (5, 22, 23, 26). Our data showed that while the parental erlotinib-sensitive SCC-S cells displayed characteristics of typical epithelial cells, including expression of E-cadherin and absence of vimentin, while resistant SCC-R cells displayed a mesenchymal phenotype manifested by loss of E-cadherin and acquisition of vimentin (FIG. 4A). In addition, examination of the head and neck, bladder, and lung (FIG. 4A) cancer cell lines used in this study demonstrated a clear association of EMT markers and erlotinib sensitivity. Since mesenchymal-like cells generally expressed higher levels of Mig6 than epithelial-like cells, we next explored whether Mig6 and EGFR activity are altered during EMT. Select epithelial cell lines were treated with TGF-β1 or TGF-β3. These cell lines included 2 head and neck (SCC-S and JHU011), 2 bladder (ScaBER and 5637), and 2 NSCLC cancer cell lines (358, H226). JHU01.1 cells were highly sensitive to TGF-β induced apoptosis and therefore could not be further studied (data not shown), while SCC-S, ScaBER, 5637 and H226 could not be induced to an overt mesenchymal phenotype after up to 14 days of treatment (data not shown). However, EMT was successfully induced in 358 cells. Examining the EMT markers E-cadherin and vimentin after TGF-β1 and TGF-β3 treatment for 1 day, 3 days, 7 days, 14 days and 21 days, we observed an overt transition of 358 cells by day 7, with a complete transition seen by day 14 (complete loss of E-cadherin) (FIG. 4B). Strikingly, both total EGFR and phospho-EGFR were reduced concomitantly with the transition, with phospho-EGFR almost completely lost in the mesenchymal phenotype cells (FIG. 4B). The proportionately greater loss of EGFR activity than total EGFR was accompanied by elevated expression of Mig6. Concomitant with these molecular alterations, the mesenchymal-like cells acquired increasing resistance to erlotinib (FIG. 4C). In addition, we found a significant increase in AKT activity and a mild increase of phospho-ERK1/2 after EMT, with the time course of AKT activation matching that of Mig6 upregulation (FIG. 4D). To further confirm the role of AKT in upregulating Mig6, we treated 358, 358/TGFβ1 day 21, and 358/TGFβ3-day 21 cells with LY294002 (PI3K inhibitor), U0126 (MEK inhibitor) or erlotinib. In 358 cells, all three inhibitors reduced the expression of Mig6, suggesting that basal EGFR activity plays a significant role in maintaining Mig6 expression (FIG. 4E). In 358/TGFβ1-day 21 and 358/TGFβ3-day 21 cells, however, while LY294002 produced the most significant inhibition of Mig6, EGFR inhibition by erlotinib failed to suppress Mig6 expression (FIG. 4E). Taken together, these data indicate that Mig6 elevation in EMT cells is due to enhanced AKT activity which comes from EGRF-independent tyrosine kinase. Enhanced Mig6 expression independent of EGFR ensures sufficient abundance to suppress basal EGFR activity during kinase switching in mesenchymal-like cells. It is noteworthy that activation of EGFR by treating these cells with EGF still upregulates Mig6, which suggests that the EGFR pathway remains functional in regulating Mig6 (FIG. 4E). A recently published EMT model using 358 cells similar to what we describe here confirmed that TGF-β-induced mesenchymal-like 358 cells exhibit aberrant PDGFR and FGFR expression, and that autocrine signaling through these receptors can activate the MEK-ERK and PI3K pathways. Similar aberrant expression of PDGFR and FGFR, and even IGFR and their ligands have also been observed by microarray analysis in our SCC-R cells. These data suggest that loss of EGFR-independence and subsequent kinase switching was made possible by upregulation of Mig6 through these other RTKs, and once the cells switch kinases for survival and proliferation, they become indifferent to EGFR inhibition.

### Example 6

### Mig6 Expression is Associated with Erlotinib Sensitivity in Directly Xenografted Human Lung and Pancreatic Tumors

To investigate whether our observations with tumor cell lines could be validated in tumor samples from patients, we analyzed directly xenografted low passage human tumors that have been shown to retain the key features of the original tumor, including drug sensitivity, and that accurately represent the heterogeneity of the disease (27). We Obtained 4 human NSCLCs, and 18 pancreatic tumors that were directly xenografted into nude mice (17). No erlotinib-sensitizing mutations in EGFR were detected in any of these tumors. We initially tested the response of the 4 patient-derived lung xenografts (BML-1, BML-5, BML-7 and BML-11) to erlotinib. Among them, BML-5 showed a better response to erlotinib than the other 3 tumors (FIG. 5A). Analysis of Mig6 expression in tumor xenografts showed that BML-1 and BML-5 expressed less Mig6 than BML-7 and BML-11 (FIGS. 5B and C). In addition, BML-5 expressed higher total EGFR as well as higher basal EGFR phosphorylation than the other tumors (FIGS. 5B and C).

We next characterized and plotted erlotinib responsiveness of 18 directly xenografted pancreatic tumors. Tumor growth inhibition data are displayed with the most sensitive tumors on the far left and the most resistant on the far right (FIG. 5D). Tumor characteristics, including KRAS mutation status as well as EGFR expression and phosphorylation levels, have been reported previously (17, 18). No EGFR mutation was found in any of these tumors. EGFR negative tumors tended to cluster on the right side of the map, indicating that they were more resistant to erlotinib. However, in EGFR-positive tumors we saw little association between erlotinib sensitivity and EGFR expression (FIG. 5D). Instead, we found that as Mig6 expression increased, tumors exhibited a more erlotinib-resistant phenotype. For example, the erlotinib-resistant tumor PANC420 expressed markedly higher Mig6 than the erlotinib-sensitive tumor PANC410, even though they expressed comparable amounts of EGFR protein (17, 18). In keeping with their Mig6 expression status, PANC410 displayed heavy EGFR phosphorylation whereas PANC420 harbored no detectable EGFR phosphorylation (17, 18). Interestingly, in the 3 erlotinib-resistant pancreatic tumors studied that displayed significantly lower Mig6 expression (PANC140, 294, and 215), IHC labeling revealed that 2 of these 3 xenograft lines did not express EGFR (17). These exceptions in situations of low or absent EGFR expression are consistent with our findings in passaged tumor lines.

### Example 7

### Mig6/EGFR Ratio Correlates with the Response of Patients to Iressa

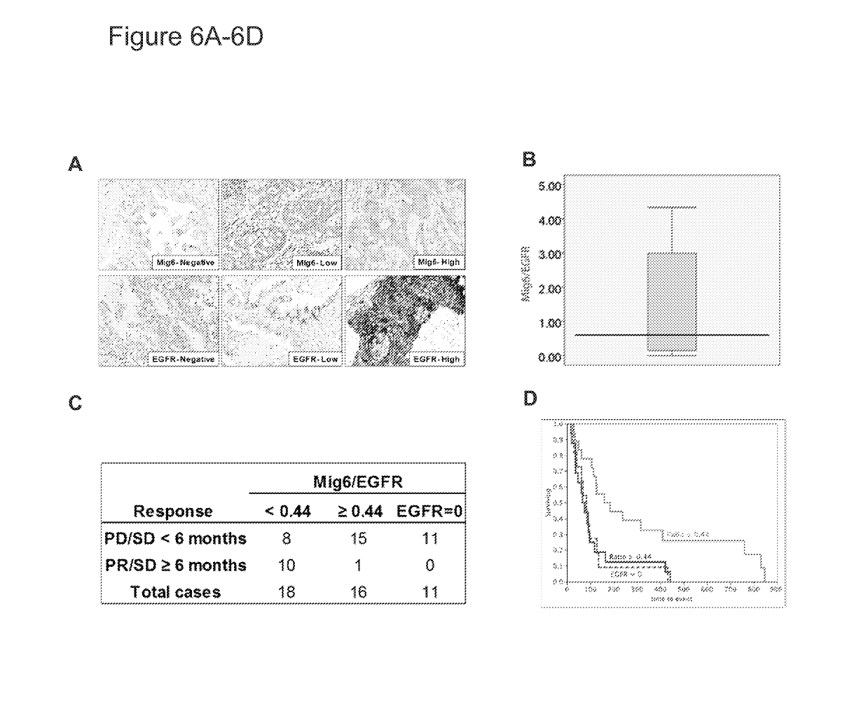

To investigate whether relative levels of Mig6 and EGFR expression correlate with the clinical drug response to anti-EGFR TKIs, we examined Mig6 and EGFR expression immunohistochemically and in blinded fashion on tissues from a cohort of lung cancer patients who had previously been treated prospectively with gefitinib alone (FIG. 6A). Mig6 cytoplasmic expression and EGFR membranous expression were analyzed in tumor cells using a score calculated using intensity (0-3+) multiplied by extension of expression (0-100%; range 0-300). Expression ratios were calculated as Mig6 expression/EGFR expression (ratios ranged from 0 to 4.33, FIG. 6B). We grouped the patients with positive EGFR (>0) staining in low or high Mig6/EGFR ratio groups using the number close to median (0.44) as the cutoff. Our data showed that the 2 patients who had partial response (PR) were in the low ratio group, with ratios of 0 and 0.14 (Table 1). In addition, patients with lower Mig6/EGFR ratio have significant better outcome than the rest of the patients (Fisher exact test, P=0.002, FIG. 6C). Kaplan-Meier survival curves showed that patients with a low Mig6/EGFR ratio survived statistically significantly longer than the high ratio patients and EGFR negative patients (FIG. 6D, Log-Rank test P=0.01). The median progression-free survival (PFS) was 96 days for the entire cohort, 71 days for high ratio group, and 83 days for EGFR negative group. However, the median PFS in low ratio group was 172 days, approximately 100 days longer than patients in either the high or EGFR negative groups. These data suggest that patients whose tumors express lower Mig6/EGFR ratio were much more responsive to Iressa treatment. The statistical significance of this comparison was sensitive to the choice of cutpoint for the ratio, so it must be considered exploratory until a prospective trial is carried out using this ratio.

TABLE 1
Summary of the clinical and pathological information of 45 patients
with advanced non-small cell lung carcinoma included in this study.

| | Mig6/EGFR | | |
|---|---|---|---|
| | <0.44 | ≧0.44 | EGFR = 0 |
| Covariate | (n = 18) | (n = 16) | (n = 11) |
| Age, mean, years | 57.4 | 61.9 | 59.6 |
| Sex | | | |
| Female (n = 25) | 7 | 10 | 8 |
| Male (n = 20) | 11 | 6 | 3 |
| Race | | | |
| Asian (n = 4) | 0 | 2 | 2 |
| Caucasian (n = 34) | 14 | 13 | 7 |
| Other (n = 7) | 4 | 3 | 4 |
| Smoking Status | | | |
| Never (n = 11) | 1 | 4 | 6 |
| Former (n = 19) | 10 | 7 | 2 |
| Current (n = 15) | 7 | 5 | 3 |
| Histology | | | |
| Adenocarcinoma (n = 31) | 2 | 11 | 18 |
| Squamous cell carcinoma (n = 10) | 5 | 1 | 4 |
| Large cell carcinoma (n = 1) | 0 | 1 | 0 |
| Adenosquamous carcinoma (n = 1) | 1 | 0 | 0 |
| NSCLC (n = 2) | 2 | 0 | 0 |
| KRAS mutation† | | | |
| No (n = 33) | 16 | 10 | 7 |
| Yes (n = 9) | 2 | 6 | 1 |
| EGFR mutation* | | | |
| No (n = 40) | 17 | 15 | 8 |
| Yes (n = 3) | 1 | 1 | 1 |
| Disease progression | | | |
| Progessive disease (n = 26) | 5 | 12 | 9 |
| Stable disease <6 mo (n = 8) | 3 | 3 | 2 |
| Stable disease ≧6 mo (n = 9) | 8 | 1 | 0 |
| Partial response (n = 2) | 2 | 0 | 0 |

†KRAS mutation information was available in 42 cases
*EGFR mutation information was available in 43 cases

### REFERENCES

The disclosure of each reference cited is expressly incorporated herein.
1. T. J. Lynch et al., N Engl J Med 350, 2129 (May 20, 2004).
2. M. S. Tsao et al., N Engl J Med 353, 133 (Jul. 14, 2005).
3. W. Pao et al., PLoS Med 2, e73 (March, 2005).
4. J. G. Paez et al., Science 304, 1497 (Jun. 4, 2004).
5. J. A. Engelman et al., J Clin Invest 116, 2695 (October, 2006).
6. Y. Lemos-Gonzalez et al., Tumour Biol 28, 273 (2007),
7. C. W. Tzeng et al., Surgery 141, 464 (April, 2007).
8. M. J. van den Bent et al., J Clin Oncol 27, 1268 (Mar. 10, 2009).
9. H. S. Parra et al., Br Cancer 91, 208 (Jul. 19, 2004).
10. F. Cappuzzo et al., J Natl Cancer Inst 97, 643 (May 4, 2005),
11. L. Fiorentino et al., Mol Cell Biol 20, 7735 (October, 2000).
12. P. O. Hackel, M. Gishizky, A. Ulrich, Biol Chem 382, 1649 (December, 2001).
13. S. Anastasi et al., Oncogene 22, 4221 (Jul. 3, 2003).
14. X. Zhang et al., Nature 450, 741 (Nov. 29, 2007).
15. I. Ferby et al., Nat Med 12, 568 (May, 2006).
16. S. Benavente et al., Clin Cancer Res 15, 1585 (Mar. 1, 2009),
17, A. Jimeno et al., Cancer Res 68, 2841 (Apr. 15, 2008).
18. H. C. Harsha et al., Proteome Res 7, 4651 (November, 2008).
19. E. Massarelli et al., Clin Cancer Res 13, 2890 (May 15, 2007).
20. W. D. Travis, Semin Respir Crit Care Med 25, 475 (October, 2004).
21. P. Therasse et al., J Natl Cancer Inst 92, 205 (Feb. 2, 2000).
22. J. A. Engelman et al., Science 316, 1039 (May 18, 2007).
23. F. Yamasaki et al., Cancer Res 67, 5779 (Jun. 15, 2007).
24. M. Fiorini et al., Oncogene 21, 6530 (Sep. 19, 2002).
25. M. Hidalgo et al, J Clin Oncol 19, 3267 (Jul. 1, 2001).
26, D. A. Haas-Kogan et al. J Natl Cancer Inst 97, 880 (Jun. 15, 2005),
27. A. Jimeno et al., Mol Cancer Ther 6, 1079 (March, 2007).
28. S. Thomson, F. Petti, I. Sujka-Kwok, D. Epstein, J. D. Haley, Clin Exp Metastasis 25, 843 (2008).
29. S. Zhang et al., Nat Med (March 13).
30. M. Guix et al., J Clin Invest 118, 2609 (July, 2008).
31. D. L. Wheeler et al. Oncogene 27, 3944 (Jun. 26, 2008),
32. R. Sordella, D. W. Bell, D. A. Haber, J. Settleman, Science 305, 1163 (Aug. 20, 2004).
33. R. L. Yauch et al., Clin Cancer Res 11, 8686 (Dec. 15, 2005).
34. S. Thomson et al., Cancer Res 65, 9455 (Oct. 15, 2005).
35. B. A. Frederick et al., Mol Cancer Ther 6, 1683 (June, 2007).
36. E. Buck et al., Mol Cancer Ther 6, 532 (February, 2007).
37. M. Shrader et al., Mol Cancer Ther 6, 277 (January, 2007).
38. A. Descot et al., Mol Cell 35, 291 (Aug. 14, 2009),
39. L. Adam et al., Clin Cancer Res 15, 5060 (Aug. 15, 2009).
40. Y. Y. Janjigian et al., Clin Cancer Res (January 19).
41. M. Fukuoka et al., J Clin Oncol. 21, 2237 (Jun. 15, 2003).

1. A method of predicting tumor resistance to an epidermal growth factor receptor (EGFR) tyrosine kinase inhibitor, comprising:
testing a patient tumor sample and determining expression level of mitogen inducible gene 6 (Mig6) and of EGFR in the sample; and
comparing the expression level of mitogen inducible gene 6 (Mig6) to the expression level of EGFR, wherein a ratio of Mig6 to EGFR lower than a predetermined cut-off value indicates sensitivity to the EGFR tyrosine kinase inhibitor and a ratio of Mig 6 higher than the predetermined cut-off value indicates resistance to the EGFR tyrosine kinase inhibitor.

2. The method of claim 1 wherein the predetermined cut-off value is 0.44.

3. The method of claim 1 wherein the expression level determined is of protein expression.

4. The method of claim 1 wherein the expression level determined is of mRNA expression.

5. The method of claim 1 wherein the tumor is selected from the group of tumors consisting of lung, bladder, head and neck, and pancreatic tumors.

6. The method of claim 1 wherein the EGFR tyrosine kinase inhibitor is erlotinib.

7. The method of claim 1 wherein EGFR tyrosine kinase inhibitor is gefitinib.

8. The method of claim 1 wherein the inhibitor is vandetanib.

9. The method of claim 1 wherein the ratio is lower than the predetermined cut-off value, and the tumor is identified as sensitive to EGFR tyrosine kinase inhibitors.

10. The method of claim 9 wherein the EGFR tyrosine kinase inhibitor is erlotinib.

11. The method of claim 9 wherein the EGFR tyrosine kinase inhibitor is gefitinib.

12. The method of claim 9 further comprising prescribing erlotinib to the patient.

13. The method of claim 9 further comprising prescribing gefitinib to the patient.

14. The method of claim 9 further comprising administering erlotinib to the patient.

15. The method of claim 9 further comprising administering gefitinib to the patient.

16. The method of claim 3 wherein Mig6 and EGFR expression levels are tested and determined by immunohistochemistry.

17. The method of claim 1 wherein the EGFR in the patient tumor sample is wild type EGFR.

18. A method of predicting tumor resistance to an antibody to epidermal growth factor receptor (EGFR), comprising:
testing a patient tumor sample and determining expression level of mitogen inducible gene 6 (Mig6) and of EGFR; and
comparing the expression level of mitogen inducible gene 6 (Mig6) to the expression level of EGFR, wherein a ratio of Mig6 to EGFR lower than a predetermined cut-off value indicates sensitivity to the antibody and a ratio of Mig 6 higher than a predetermined cut-off value indicates resistance to the antibody.

19. The method of claim 18 wherein the predetermined cut-off value is 0.44.

20. The method of claim 18 wherein the expression level determined is of protein expression.

21. The method of claim 18 wherein the expression level determined is of mRNA expression.

22. The method of claim 18 wherein the tumor is selected from the group of tumors consisting of lung, bladder, head and neck, and pancreatic tumors.

23. The method of claim 18 wherein the antibody is cetuximab.

24. The method of claim 18 wherein the antibody is panitimumab.

25. The method of claim 18 wherein the EGFR in the patient tumor sample is wild type EGFR.

26. The method of claim 20 wherein Mig6 and EGFR expression levels are tested and determined by immunohistochemistry.

27. The method of claim 1 or 18 wherein the patient tumor sample is a surgically dissected tumor.

28. The method of claim 1 or 18 wherein the patient tumor sample is a biopsy.

29. The method of claim 1 or 18 wherein the patient tumor sample is a xenografted, low passage human tumor.

30. The method of claim 1 or 18 wherein at least two patient tumor samples from a patient are tested and expression levels determined, wherein the patient tumor samples are obtained at distinct times, wherein an increase in the ratio over time indicates an increase in resistance.

31. A method of stratifying patients on the basis of tumor characteristics, comprising:
testing a patient tumor sample and determining expression level of mitogen inducible gene 6 (Mig6) and of EGFR;
comparing the expression level of mitogen inducible gene 6 (Mig6) to the expression level of EGFR; and
assigning the patient to a first group if a ratio of Mig6 to EGFR lower than a predetermined cut-off value is determined and assigning the patient to a second group if a ratio higher than a predetermined cut-off value is determined.

32. The method of claim 31 wherein the first and second groups are subjected to a clinical trial.

33. The method of claim 31 wherein the first group is a group which is treated with an EGFR inhibitor selected from the group consisting of an anti-EGFR antibody and a tyrosine kinase inhibitor.

34. The method of claim 31 wherein the predetermined cut-off value is 0.44.

35. The method of claim 31 wherein the expression level determined is of protein expression.

36. The method of claim 31 wherein the expression level determined is of mRNA expression

37. A method of predicting tumor resistance to an inhibitor of epidermal growth factor receptor (EGFR) selected from the group consisting of an anti-EGFR antibody and a tyrosine kinase inhibitor, comprising:
testing a patient tumor sample isolated from a patient at a first time and determining expression level of mitogen inducible gene 6 (Mig6);
testing a patient tumor sample isolated from a patient at a second time, later than the first time, and determining expression level of mitogen inducible gene 6 (Mig6); wherein an increase in the expression level of Mig6 over time indicates an increase in the resistance of the tumor to the inhibitor.

38. The method of claim 37 wherein the expression level determined is of protein expression.

39. The method of claim 37 wherein the expression level determined is of mRNA expression.
